(19) United States
(12) Patent Application Publication
(10) Pub. No.: US 2005/0038488 A1
(43) Pub. Date: Feb. 17, 2005

(54) X-ray apparatus with field emission current stabilization and method of providing x-ray radiation therapy

(76) Inventors: Ali Jaafar, Eden Prairie, MN (US); Victor I. Chornenky, Minnetonka, MN (US)

Correspondence Address: OFFICES OF CRAIG GREGERSEN, P.O. BOX 386353, 10032 QUEBEC AVENUE SOUTH, BLOOMINGTON, MN, 55438, US

(21) Appl. No.: 10/938,971

(22) Filed: Sep. 10, 2004

Related U.S. Application Data
(63) Continuation-in-part of application No. 10/392,167, filed on Mar. 19, 2003.

(51) Int. Cl. A61F 2/00

(52) U.S. Cl. 607/100

(57) ABSTRACT

The present invention provides apparatus and method for providing a stabilized x-ray output from a field emission x-ray apparatus by monitoring the operating current and adjusting the gap between the anode and cathode to stabilize the output.

The present application claims priority from U.S. patent application Ser. No. 10/392,167, entitled “X-Ray Apparatus With Field Emission Current Stabilization And Method Of Providing X-Ray Radiation Therapy” and filed on Mar. 19, 2003.

The present invention relates generally to apparatus and method for providing x-ray radiation therapy and specifically to apparatus and method for providing x-ray radiation therapy with real-time stabilization of the operating current, and thus the dosage rate.

### BACKGROUND OF THE INVENTION

The use of x-ray radiation for medical and non-medical applications is well known. In the medical arena, x-ray radiation therapy is a commonly used and accepted practice in the treatment of disease, including but not limited to, for example, tumors, certain skin diseases, and/or benign conditions. Historically, treatment first utilized external x-ray sources that supplied x-ray radiation to the target site. Where the target site was internal, such as a tumor, the applied x-ray radiation had to traverse the skin and other soft tissue and perhaps bone on its way to the target site, resulting in damage and burn to those tissues. Among other reasons, this disadvantage of x-ray therapy using external x-ray sources caused innovators to seek devices and methods to generate x-rays internally.

Generally speaking, there are two basic types of x-ray equipment in use today. One type relies upon heating an electron source to generate thermionically a beam of electrons that are then directed across a vacuum gap to a target material such as gold or tungsten or other high atomic number material. X-rays are generated upon the beam striking the target. In the second type, known as a field emission emitter, an electric field pulls electrons from a cathode across a vacuum gap toward an anode to strike a target material and generate the x-ray radiation. In both types, the generated electron beam is directed through a high vacuum to avoid electric breakdown and dissipation of the electron beam—and a subsequent reduction in the beam intensity—by atoms in the gap.

X-ray emitters for medical and non-medical applications take many forms. For example, one known type of emitter uses an x-ray source for intracavitary irradiation. The source comprises a housing, an elongated tubular probe, a target assembly, and an inflatable balloon. The housing encloses a thermionic electron gun and includes elements for directing the electron beam, generated in the housing, into the tubular probe. The tubular probe extends along a central axis from the housing about the beam path. The target assembly extends along the central axis and is coupled to the end of the probe distal from the housing. The target assembly includes a target element positioned in the beam path, and adapted to emit x-rays in response to the impinging electron beam. The balloon is affixed to the distal end of the probe and is inflatable so that when that probe end is inserted into a body cavity, the balloon may be inflated to stretch the cavity to a known shape.

The previously described apparatus has several drawbacks. First, the x-ray system has an inherent instability of its electron beam in the presence of a magnetic field. Because the thermionically generated electron beam must traverse the length of the probe between the electron gun and the target assembly, stray external magnetic fields can cause the beam to be deflected away from the target causing the generated x-ray flux to vary and complicating the calculation of the dose actually received by the patient. To address this drawback, the system requires an additional system for controlling the beam direction. Another drawback is that the apparatus includes an electron gun, which significantly adds both complexity and cost. Still another deficiency in this system is that the inflated balloon does not fix the position of the x-ray source relative to the patient's body and thus it requires an additional system for ensuring that the x-ray emitter is in the right position against the tissue to be irradiated.

Another x-ray device uses an X-ray needle for interstitial radiation treatment, This device includes an elongated X-ray tube coupled to an electron gun at one end of the tube, and a converter element converting the energy of electrons into the X-ray energy, disposed at the other end of the tube. The x-ray source comprises a solenoid coil wound around the tube for providing a magnetic field that confines the emitted electrons within a narrow beam. An elongated outer casing encloses the tube and coil. The x-ray source also includes a cooling system for removal of the heat generated by the converter and the magnetic coil. The drawbacks of the disclosed X-ray source are its relative complexity, large size and lack of adequate means for delivery of an optimal distribution of radiation dose across the predetermined volume of the target tissue.

Another known x-ray device utilizes a miniature X-ray tube with a direct current power supply and a field emission cathode. The tube has a needle cathode along its axis and an exit window at the end of the tube behind the cathode. The tube generates x-ray radiation along the axis of the device. It is not adapted for and cannot be used for treatment of tumors inside the body. Another drawback of the x-ray tube is an absence of the ability to control the operating current and voltage independently. This particular disadvantage inhibits manufacturing reproducibility.

In using x-rays for medical therapy it is important that the proper dose rate be applied. The dose depends upon the energy of the x-rays and the intensity of the x-ray beam. In field emission devices, increasing the voltage of the electric field increases the energy of the x-rays while increasing the current increases the intensity of the beam. Higher energy x-rays penetrate to greater depths in body tissue, so voltage control is important in controlling the energy to avoid damaging healthy tissue needlessly due to an undesired depth of penetration of the x-rays. The beam flux is also dependent upon the gap between the anode and the cathode. Increasing the gap decreases the beam flux and vice versa.

An undesirable feature of known field x-ray emitter devices is the inability to closely control the dose rate. One reason for this lack of control is that the generation of the electron beam from the cathode can be sporadic. That is, due to uncontrollable changes in the condition of the electron emitting surface of the cathode, field emitters are known for instability of their current, which can vary by a factor of 2. At the higher end of this range the emission current can overheat the emission site and create a vacuum discharge over the gap that can significantly change the electric properties of the gap or even make the device inoperable in the required settings. Because of the inconsistency in the current and thus the x-ray beam flux, the dose applied during any particular therapy session may not be well known, which leads to inconsistent treatment and results. The only sure way to know that a particular medical problem has been adequately addressed is to apply radiation at a presupposed rate that increases the likelihood of damage to healthy tissue.

There is a need for an apparatus and method that enables an operator of an x-ray apparatus to control the energy and intensity of an emitted x-ray beam by independently controlling the voltage and operating current, respectively. It would be desirable to have such an apparatus and method for use in standard operating rooms, which cannot currently be used where irradiation is supplied by widely used naturally occurring radioactive isotopes such as iridium 192 because of a lack of protection from the highly penetrating radiation produced by such sources. It would also be desirable to have an x-ray device that is not sensitive to the external magnetic field in the manner of x-ray sources using an electron gun. Additionally, it would be desirable for such an apparatus and method to provide a low cost source of ionizing radiation for radiation brachytherapy of brain, breast, prostate and other tumors or for radiation brachytherapy of non-tumor related medical problems.

### BRIEF DESCRIPTION OF THE INVENTION

The present invention provides an apparatus and method for radiation therapy that enables the operator to exercise independent control of the voltage and operating current, thus providing the operator with the ability to stabilize the applied radiation dose supplied to the target site. An apparatus in accord with the present invention will have a field emission cathode that produces an electron beam in response to an applied operating voltage and an anode having a target material that generates x-rays when struck by the electron beam. The cathode and anode are separated by a gap changeable in size in response to the x-ray output of the device to maintain the dose at the desired level.

A method in accord with the present invention will involve steps of identifying a target site for radiation therapy; disposing a field emission x-ray apparatus having a cathode and an anode separated by a gap in proximity to the target site; monitoring the operating current of the x-ray apparatus; and adjusting the gap to maintain the desired operating current. Adjusting the gap enables the operator to control the operating current, thereby enabling compensation for possible instabilities in the field emission of electrons, including but not limited to instabilities caused by the state of the cathode emission surface, drift of operating parameters with time, and temperature.

In an embodiment of the present invention, an x-ray apparatus may have a vacuum housing and a probe attached thereto. The probe may have an elongated, tubular or needle-like configuration. The distal end of the probe may have a heavy-metal anode and a field emission cathode separated by a vacuum gap, the anode and cathode being provided for production of x-rays when an operating voltage is applied between them. Independent control of the operating current is provided to the operator by the inclusion of an adjustment mechanism for adjusting the gap size. In an embodiment of the invention, the adjustment mechanism may take the form of a linear translator.

For delivery of a predetermined radiation dose, the distal end of the probe is introduced into the body in proximity of the previously identified target or treatment site and the operating voltage is applied over a predetermined period of time. For optimal distribution of radiation along the treatment area a pullback mechanism may be provided that allows the operator to step-wise position and, if desired, rotate the probe during a radiation therapy procedure.

The cathode is adapted to emit electrons when an operating voltage is applied between the electrodes. As the electrons, emitted by the cathode, impinge on the anode, the x-rays are radiated in a predetermined spatial pattern. The irradiation pattern may vary for different implementation of the device. The depth of penetration of x-ray radiation in tissue is defined by the operating voltage and is predetermined for the procedure.

The present invention further contemplates temperature dissipation apparatus to dissipate heat generated by the creation and emission of x-rays, wherein such temperature dissipation or cooling apparatus includes a fluid pump for pumping a cooling fluid and a cooling jacket enclosing the probe that receives the cooling fluid from the pump and returns it to the pump.

The present invention further contemplates the use of a tissue stretching appliance to enlarge and provide a desired symmetrically configured cavity at the location of an excised tumor to facilitate irradiation of the margin tissue surrounding the excision cavity.

The present invention, as well as its various features and advantages, will become evident to those skilled in the art when the following description of the invention is read in conjunction with the accompanying drawings as briefly described below and the appended claims.

### BRIEF DESCRIPTION OF THE DRAWINGS

FIG. 1 illustrates an embodiment of the present invention in a partial cross sectional, partial schematic plan view.

FIGS. 2a-2d illustrate different alternate embodiments of the distal end of a probe of an x-ray apparatus in accord with the present invention.

FIG. 3 depicts another alternate embodiment of the distal probe end of an x-ray apparatus in accord with the present invention.

FIG. 4 schematically illustrates an x-ray apparatus in accord with the present invention and including a balloon and a pull-back mechanism.

FIG. 5 schematically illustrates another embodiment of an x-ray apparatus in accord with the present invention.

FIG. 6 schematically shows another embodiment of an x-ray apparatus in accord with the present invention wherein an inchworm linear motor is used to adjust the gap size.

FIG. 7 illustrates another embodiment of the present invention wherein the probe comprises a material of high dielectric strength with a conductive outer coating.

FIG. 8 illustrates another embodiment of the present invention for providing a generally omni-directional x-ray distribution pattern.

FIG. 9 depicts another embodiment of the present invention wherein an insulating layer between the cathode needle and the surrounding probe body is provided by a material of high dielectric strength spatially disposed form the needle and the probe body.

FIG. 10a illustrates an embodiment of the present invention including apparatus for dissipation of the heat generated during operation thereof.

FIG. 10b is a cross-sectional view of the embodiment of FIG. 11a taken along viewing plane A-A.

FIG. 11a depicts an appliance in its collapsed position for stretching a body cavity created by the removal of a tumor.

FIG. 11b depicts an appliance in its expanded position for stretching a body cavity created by the removal of a tumor.

FIG. 12 shows an embodiment of the present invention including an apparatus for stretching a body cavity.

FIG. 13 illustrates the embodiments of FIGS. 11 and 12 in use with the present invention intra-operatively in a body cavity.

### DETAILED DESCRIPTION OF THE INVENTION

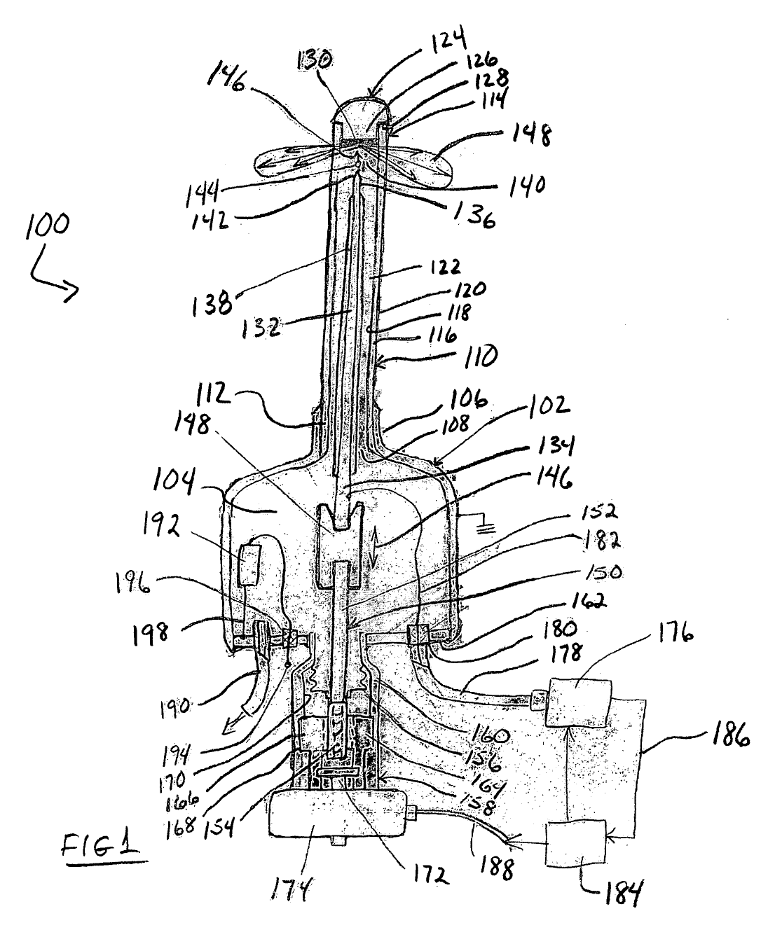

An embodiment of an x-ray apparatus with gap size control 100 is shown in FIG. 1. Apparatus 100 includes a housing 102 that defines a vacuum chamber 104. Housing 102 can take substantially any desired form and as shown has a substantially cylindrical configuration. Housing 102 may include a collar 106 that may, if desired and as shown, be integral with the housing 102. Collar 106 forms an opening 108 into the housing 102 that is configured to receive a probe 110 having proximal and distal ends 112 and 114, respectively. Collar 106 receives proximal end 112 in a sealing engagement to preserve the vacuum within housing 102. While a protruding collar 106 is illustrated in the Figure, other known forms of sealing engagements and configurations therefor can be used with equal facility in the present invention so long as a vacuum can be maintained.

Probe 110 may have an elongate, tubular or needle-like configuration as shown in the Figure. It will be understood that while the embodiments of a probe used in association with the present invention shown herein will be described as being tubular or needle-like, that such descriptions are exemplary and that other shapes, if useful for a particular procedure, could also be used with the present invention. Thus, probe 110 includes an outer wall structure 116, here a cylindrical wall 116 having inner and outer surfaces 118 and 120, respectively, that defines a hollow interior 122 that communicates with vacuum chamber 104, and is thus also maintained as a vacuum, through its open proximal end 112. The other open, distal end 114 is sealingly enclosed by an anode electrode 124, which forms an end cap for the probe 110. Anode 124 includes an inwardly projecting mating portion 126 that is received within the probe 110 and a shoulder 128 that engages the end of the probe 110. Anode 124 can be sealingly attached to the probe 110 in any known manner, such as by brazing. In one embodiment of the present invention, anode 124 may be made of aluminum and may have a thin layer (0.25-0.5 microns) of gold, tungsten, or other known heavy metal, 130 deposited onto the anode surface.

A field emission cathode electrode 132 is disposed substantially within probe interior 122. Cathode 132 has proximal and distal ends 134 and 136. The cathode 132 is disposed substantially centrally within the probe 110 along its longitudinal axis, thus avoiding contact with the probe wall 116. Cathode 132 is preferably clad in an insulating layer 138 to prevent a high voltage electric breakdown between the cathode 126 and the probe 110, which is connected to the anode 124, during operation of the apparatus 100. Insulating layer 132 is preferably made from a high dielectric strength material.

Cathode 132 is spaced apart from anode 124 by a gap 140. When an operating voltage is applied across the gap 140 between the cathode 132 and the anode 124, the tip 142 of the cathode 132 emits electrons 144 (shown greatly exaggerated in size for purposes of illustration only) that travel across the gap 140 to the anode 124, as indicated by directional arrow 146. The radius of curvature of the sharp tip 142 is in a range of several tens of micrometers. As the electrons 144, emitted by the cathode 132, impinge on the anode 124, x-rays are radiated by the anode in a spatial pattern 148.

The depth of penetration of x-ray radiation emitted by anode 124 into tissue is defined by the applied operational voltage. During a radiation therapy procedure, a selected operating voltage is applied as previously discussed and the field emission cathode 132 starts emitting electrons 144, thus creating an operating current through the vacuum gap. The magnitude of this current depends in part on the size of the vacuum gap 140. As noted previously, known x-ray emitters are provided with gaps of fixed size, limiting the ability of the operator to control the radiation dose received by the patient. The present invention provides an operator greater control over the radiation dose by providing apparatus and method for adjusting the gap size, as will be described further below.

Thus, as seen in FIG. 1, the proximal end 134 of the cathode 132 is attached to an insulator 148, which, in turn, is secured to a movable shaft 150 at its distal end 152. The proximal end 154 of the movable shaft 150 has a fine thread and via a flexible bellow 156 is engaged with a translational stage 158. Translational stage 158 is secured to the vacuum housing 102 with a rigid tube 160 welded into the base plate 162 of vacuum housing 102.

Translational stage 158 comprises a threaded nut 164 that threadably receives the threaded proximal end 154 of the shaft 150. The outer perimeter 166 of nut 164 is rotationally received by an appropriately configured recess 168 in the inner surface 170 of the tube 160. Nut 164 is attached to a rotor 172 of a step motor 174. Rotation of the rotor 172 by motor 174 causes nut 164 to rotate, threading the proximal end 154 of the shaft 150 into or out of the nut 164 depending on the direction of rotation of the rotor. As the shaft 150 threads into or out of the nut 164, the tip 142 of the cathode 132 moves away from or towards the anode 124, changing the size of the gap 140 and thus regulating the operating current across the gap 140. Increasing the size of the gap decreases the operating current while decreasing the gap size increases the operating current.

The operating voltage for the apparatus 100 is provided by a high voltage DC source 176, which is connected the cathode 132 by an appropriate insulated connector 178. Connector 178 extends through base plate 162 through a high voltage feed-through 180. If desired, the electrical connector extending from feed-through 180 can be an uninsulated wire 182. DC source 176 should be configured to provide operating voltage in the range of about 10 to about 50 kV across the vacuum gap 140.

During an x-ray radiation therapy procedure, a desired radiation dose, which is a function of the dose rate and the time period during which the radiation is applied, will be determined and the appropriate voltage and current will be selected to provide the desired dose rate and time of irradiation using a controller 184. As noted, because the operating current can vary due to changes in the state of the cathode surface, the present invention affords the operator the opportunity to stabilize the operating current by adjusting the gap size 140. To that end, high voltage source 176 will include an appropriate current sensor (not shown in the Figure), which sends the value of the current via a feedback loop 186 to controller 184. In response to this current signal, controller 184 will send the appropriate signal through an appropriate connector 188 to motor 174. This signal will cause motor to rotate nut 164 in the appropriate direction to adjust the gap size and the operating current accordingly. In this manner, the current selected for the procedure by the operator can be stabilized with high precision by the feedback loop at any pre-selected operating value of the current. That is, by adjusting the gap size, the operating current is stabilized such that the desired dose rate is stabilized at the predetermined value for the predetermined irradiation time period (also monitored by the controller 184 using well-known timer electronics for doing so), thereby providing the desired total radiation dose for the particular radiation therapy.

Preservation of the vacuum within the apparatus 100 is important to its proper functioning. To that end, the probe 110 may be made of aluminum, so welding the probe to the anode 124 at the shoulder 128 to seal the probe/anode connection can be made relatively easily. In addition, base plate 162 may be joined to the vacuum housing 102 and tube 160 may be joined to the base plate 162 by vacuum tight welds. The ultra high vacuum (10⁻⁷-10⁻⁹ Torr) required for operation of field emission devices generally, is achieved by a vacuum pump, not shown in the figure, which evacuates the vacuum housing 102 via a pipe 190. When the outgassing and pumping out of the vacuum chamber 104 is complete, the pipe 190 is sealed and pinched off. A getter 192 maintains the high vacuum in the vacuum housing 102 after the apparatus 100 is separated from the vacuum pump. The getter 192 can be reactivated by a low voltage current delivered by connector 194 via a feed-through 196 in base plate 162. Getter 192 can be connected to the housing 102 by an appropriate connector 198 to complete a circuit. As is known in the art, getter 192 is provided to absorbs vacuum contaminants to preserve the vacuum at the desired level.

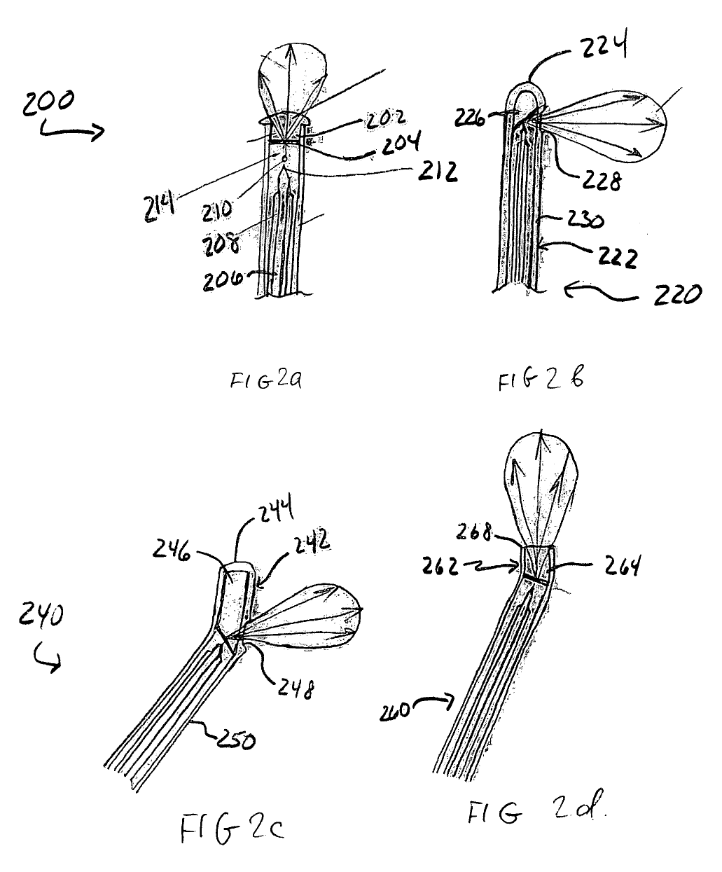

The present invention, in addition to providing dose control not found in the prior art, also can provide a variety of x-ray distribution patterns for different treatment situations. Examples of alternative embodiments of such and more detailed views of the distal end of the probe are shown in FIGS. 2a-2d. Each of the probes shown in the Figures are useful in the embodiment of the present invention illustrated in FIG. 1.

Referring to FIG. 2a, a probe distal end 200 includes an anode 202 made of beryllium vacuum sealed thereto. Anode 202 includes a thin layer 204 of heavy metal attached thereto by such known means as vapor deposition. A field emission cathode 206, clad in an insulating layer 208, emits electrons 210 (shown in greatly exaggerated detail) from the cathode tip 212 upon application of the operating current across the vacuum gap towards the anode 202. In this case, the beryllium anode 202 serves as the x-ray window and thus x-rays will be emitted in the forward direction in a substantially conical spatial pattern 216.

FIG. 2b illustrates a probe distal end 220 wherein the probe 222 has a closed end 224 with an aluminum anode 226 disposed therein. Anode 226 may also have a thin, heavy metal layer 204 deposited thereon. In this embodiment, the probe includes an x-ray window 228 in the cylindrical wall 230 of the probe 222. In this embodiment, x-rays will be emitted laterally to the longitudinal axis of the probe as indicated schematically by the spatial x-ray pattern 232. The x-ray window 228 is formed by reducing the thickness of the probe wall 230 in the desired area to facilitate the transmission of the x-rays from the probe into tissue.

FIG. 2c shows a probe 240 whose distal end 242 is angled relative to the axis of the probe, along which the cathode 206 generally lies. The distal end 242 of probe 240 has a closed end 244. A beryllium anode is disposed within the distal end of the probe. As in the embodiment shown in FIG. 2b, an x-ray window 248 is formed in the angled distal end 242 by thinning the wall 250, which made be made of aluminum, to facilitate transmission of the x-rays therethrough into the body tissue. As with the probe shown in FIG. 2c, x-rays will be emitted substantially at an angle to the axis of the probe.

FIG. 2d illustrates yet another embodiment of a probe 260. In this embodiment, a probe 260 includes an angled distal end portion 262. A beryllium anode 264 is sealingly received by the open end 266 of the angled distal end portion 262. The beryllium anode 264 will typically have a thin layer of heavy metal deposited thereon. In this embodiment, the beryllium anode 264 will serve as the x-ray window allowing transmission of the x-rays through from the probe into the tissue.

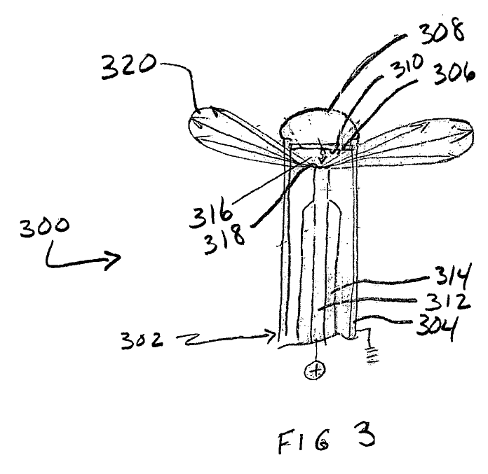

FIG. 3 illustrates another embodiment of the distal end of a probe of a field emission x-ray apparatus in accord with the present invention. Thus, as shown, an alternative embodiment of a probe 300 includes a probe distal end 302 comprising a tube 304 having an open end 306 sealingly enclosed by an end cap 308 forming a field emission cathode. Cathode 308 includes a diamond film layer 310 (or a layer of a similar material that emits electrons) that serves as the electron emitter deposited thereon. An anode 312 is centrally disposed within tube 304 and during operation is maintained at a positive potential of about 10 to about 50 kV relative to the end cap cathode 306. Like the previously discussed cathodes of FIGS. 1-2d, anode 312 is clad in an insulating layer 314 to prevent high voltage discharges between the anode and the tube 304. In this embodiment, electrons are emitted by the layer 310, traverse a vacuum gap 316 and impinge upon the blunt end 318 of the anode, causing the emission of x-rays therefrom as indicated at 320. This reversal of the relative positions of the anode and cathode provides a higher hold-off voltage for the same diameter probe than the earlier described embodiments. In addition, any heat generated at the anode is dissipated by being carried by the anode rearwardly to the back plate of the housing (such as back plate 162 shown in FIG. 1) rather than being dissipated by the outer surface of the probe tube 304 into a patient's tissue, as may occur in the embodiments of FIGS. 1-2d. Stated otherwise, this embodiment allows a manufacturer to build a smaller, needle-type probe having a diameter of about 1 mm, which does not heat the probe and can be in direct contact with the patient body. This version of the emitter may be useful for treatment of prostate cancer among other illnesses.

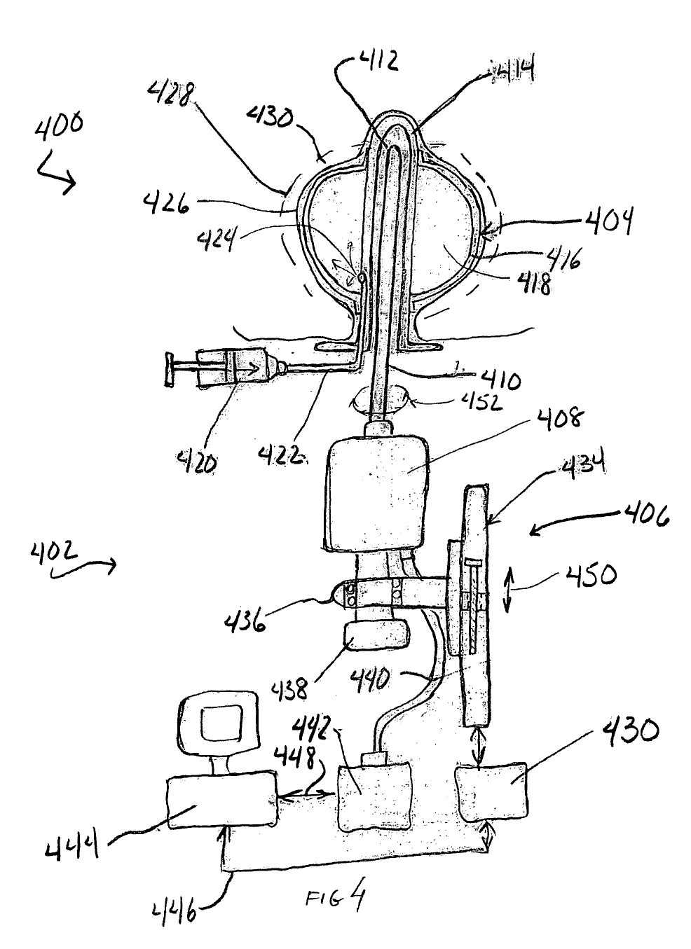

FIG. 4 schematically illustrates an x-ray system 400 for radiation brachytherapy that may find use in irradiating marginal tissue after the removal of a tumor in a brain or breast. Irradiation of a tumor site following tumor excision is desirable because it helps ensure that any cancerous cells have been either excised or killed by the radiation, thus hopefully curing the patient. System 400 comprises an x-ray emitter apparatus 402, a balloon assembly 404, and a pullback system 406. Apparatus 404 includes a housing 408 and attached probe 410 in accord with previously described x-ray emitters. Probe 410 includes at its distal end 412 an anode/cathode x-ray emitter assembly that radiates x-rays radially in all directions. Balloon assembly 404 includes a hollow shaft 414 configured to slidingly receive the probe 410. An inflatable balloon 416 is attached to the exterior of the shaft 414. Balloon 416 includes an interior volume 418 that fluidly communicates with a syringe 420 via a hollow tube 422.

During an irradiation procedure, the balloon assembly 404 will be placed within a patient at a desired therapy site, such as a cavity formed within tissue by the removal of a tumor. Inflating the balloon assembly stretches the tissue surrounding the excised tumor and provides a more uniform surface for radiation therapy. Probe 410 can be placed inside the hollow shaft 404 of the balloon assembly 404 and the balloon 416 inflated by filling it with a fluid, such as saline, that is injected by the syringe 420, travels through the tube 422 and into the balloon interior volume 418 through a tube opening 424. Alternatively, the probe can be placed within the shaft after balloon inflation and moved therealong, irradiating the marginal tissue surrounding the inflated balloon 416. In the Figure, the cavity tissue surface lying adjacent to the balloon is designated by numeral 426, the reference surface outside the cavity tissue surface (usually 1 cm off the cavity surface 410) is designated by numeral 428, and the tissue to be irradiated, know as the marginal tissue, which lies between cavity tissue surface 426 and reference surface 428, is referenced by numeral 430.

To avoid excess radiation dosage delivery to some tissue and inadequate radiation dosage delivery to other tissue surrounding the balloon 416, pullback system 406 is provided to precisely control the movement of the probe 410 within the shaft 414. Pull back systems are known in the art and will be described generally here. System 406 comprises a controller 432 and a pullback mechanism 434. Mechanism 434 includes a clamp 436 that engages an appropriately configured connector arm 438.

System 400 further includes a high voltage connector 440 extending from the x-ray emitter high voltage source 442 to the housing as described in previous embodiments of the present invention, thus providing the high voltage power source 442 to the housing 408.

A computer or other microprocessor based device 444 may be used to control the motion of the probe 410 inside the shaft 414 and the dwelling times at each point along the shaft to deliver the dose to the reference surface 428 and the marginal tissue 430 exactly as prescribed for the particular patient and the particular procedure. Computer 444 will be connected to the pullback controller 432 with the appropriate connector 446 and to the high voltage power source 442 by an appropriate connector 448. In this manner, a single computer may easily control the entire procedure, controlling the operating current as previously described and advancing and retracting the probe within the shaft 414 as indicated by arrow 450. If desired, rotational motion may also be provided by such a system 406, as indicated by rotational arrow 452, or may be provided in lieu thereof by means known to the art. The details of the pullback system 406 are well-known and have been omitted from the Figures for clarity of illustration. Pullback systems can be purchased commercially, though they may need some modifications to engage an x-ray apparatus in accord with the present invention based upon the final configuration of the apparatus, such modifications being within the skill of those versed in the art.

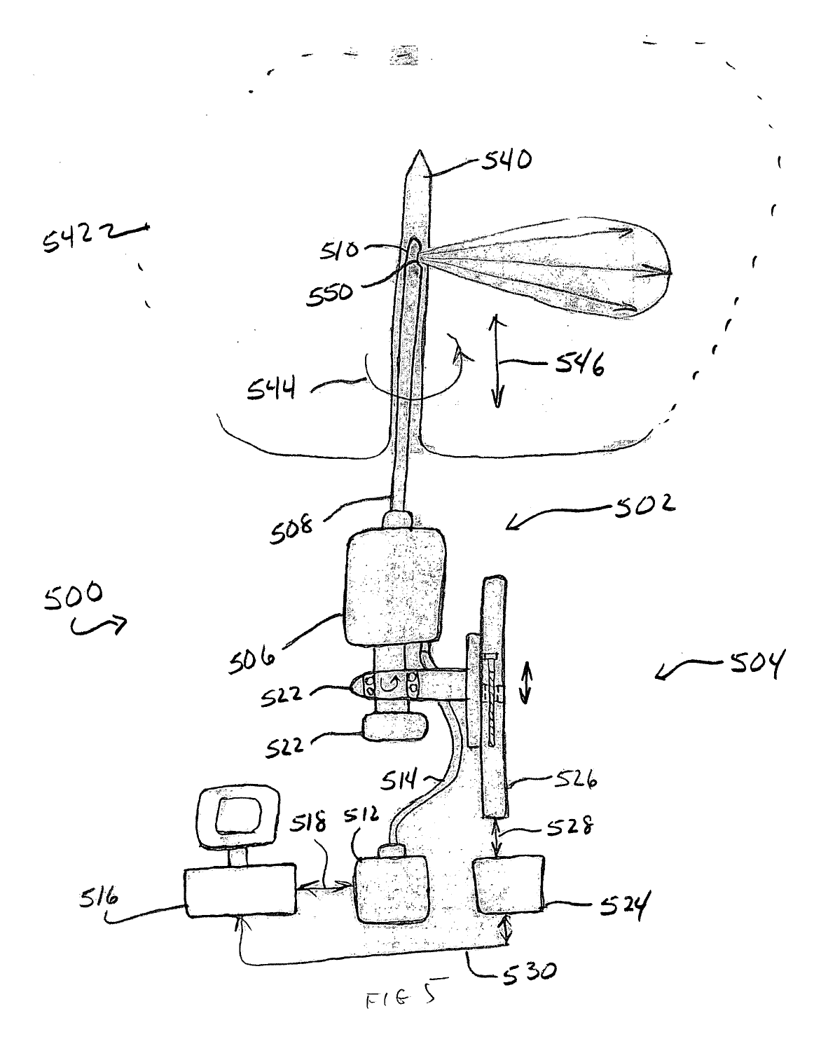

FIG. 5 illustrates another embodiment of the present invention that can be used, among other uses, where no excision of the tumor is done and no balloon is used to stretch the cavity left after the excision. In this therapy situation, the irradiation is performed on a tumor in situ in the patient's body, and may be used, for example, for treatment of prostate cancer. Thus, FIG. 5 depicts an x-ray emitter system 500 comprising an x-ray apparatus 502 and a pullback mechanism 504. The apparatus 502 will be substantially similar to the previously described x-ray apparatus and the pullback system 504 will be substantially similar to the system 406 described with reference to FIG. 4, with any differences to be noted hereafter.

Thus x-ray apparatus 502 includes a housing 506 and a probe 508 having a distal end 510. X-ray apparatus 502 will be powered by a high voltage power source 512 connected thereto by an appropriate connector 514. Control of the high voltage power source 512 is accomplished with a computer 516 or other appropriate microprocessor device through an appropriate connector 518. Pullback mechanism 504 is attached by a clamp 520 or other attachment device known in the art to a connecting member 522 attached to the housing 506. Pullback system 504 comprises a controller 524 and a pullback mechanism 526 operably connected to each other by an appropriate connector 528. Operation of the pullback mechanism can also be controlled by computer 516 via an appropriate connector 528 to pullback controller 524.

In a therapy procedure using the x-ray system 500, an elongated cavity 540 will be made in a patient's body tissue 542 with a trocar or similar surgical instrument in the vicinity of the tumor or through the tumor itself. Subsequently, probe 508 will be introduced into the cavity 540. In this embodiment of the invention, an x-ray apparatus with one side irradiation pattern is utilized, similar to that shown in FIG. 2b or FIG. 2c. Movement of the probe to provide the desired, pre-selected radiation therapy will be accomplished by the pullback system 504. System 504 will be of the type that provides rotational motion to the probe 508, as indicated by the arrow 544 as well as translational motion as indicated by arrow 546. Thus, pull system 504 will be controlled by computer 516 to dispose the probe 508, and in particular, the probe distal end 510 with its x-ray window 550 in predetermined dwelling positions for predetermined periods of times in and around the tumor while also rotating the probe 508 to provide the desired radiation exposure pattern to the tumor and surrounding tissue. Probe pullback and rotation system 504 can thus provide an asymmetric radiation pattern, thereby avoiding over-irradiation of surrounding tissues, such as the patient's urethra and rectum, as well as associated major nerves, when treating a prostate tumor, with vitally important functions. Protection of such tissues from an excessive radiation dose is important for long term success of such procedures; for example, excess irradiation of the urethra and rectum is known to be the major source of long term morbidity after an otherwise successful radiation treatment for prostate cancer.

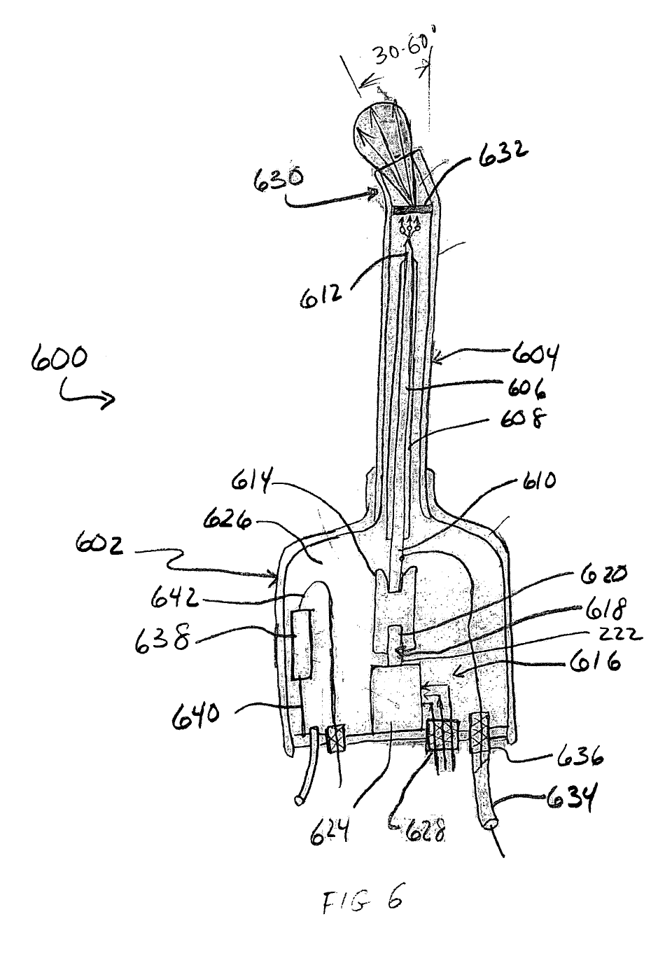

FIG. 6 shows another embodiment of an x-ray apparatus 600 with an adjustable vacuum gap between the cathode and anode. Apparatus 600 is substantially similar to the embodiment 100 shown in FIG. 1. Thus, x-ray apparatus 600 includes a housing 602 and probe 604. A cathode 606 clad in an insulating layer 608 is disposed within the housing 602 and probe 604. Cathode 604 has proximal and distal ends 610 and 612. The proximal end 610 is connected to an insulator 614, which in turn is attached to a translation stage 616 to provide adjustment of the vacuum gap. Translation stage 616 comprises a shaft 618 that is attached at one or the distal end 620 to the insulator 614 and at the other or proximal end 622 to an inchworm linear motor 624. The inchworm linear motor 624 is a piezoelectric device generally used in micro-positioning applications due to its ability to make very small and accurate motions. One commercial source of such motor 624 is EXFO, which is located in Richardson, Tex. The inchworm linear motor 624 can be placed within the vacuum chamber 626 formed within the housing 602 and controlled via a three wire vacuum feed-through 628 by an external controller (not shown in the figure). The use of the inchworm linear motor 624 allows the overall size of the vacuum housing 602 to be reduced to about 6 to about 10 cm in length and about 2 to about 3 cm in diameter.

X-ray apparatus 600 probe distal end 630 is formed similarly to that embodiment shown in FIG. 2d wherein the distal end is angled relative to the longitudinal axis of the probe and cathode 606. In this embodiment, the emitted x-ray beam is angled about 30 to about 60 degrees with the axis of the probe 604. The distal end 630 mounts the anode 632 and the location of the angle with respect to the remainder of the probe body. Anode 632 is made from beryllium and includes a heavy metal deposit on it surface that functions as the x-ray emitter as previously described with respect to other embodiments.

Apparatus 600 is electrically connected to a high voltage power source (not shown) by an appropriate electrical connector 634 that extends through a feed-through 636. In addition, a getter 638 is provided; as with the embodiment shown in FIG. 1, getter 638 is electrically connected to an appropriate power source by electrical connectors 640 and 642 to provide reactivation.

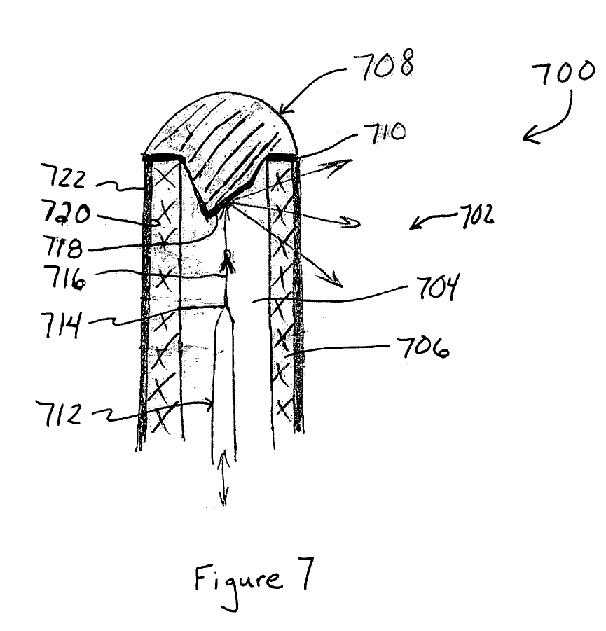

Another embodiment 700 of the present invention having a substantially radially directed x-ray pattern is depicted in FIG. 7. The Figure shows in cross section a version of the distal end of a probe 702 including a vacuum chamber 704 partly defined by a structure 706, which may be in the form of a tube, and a conductive anode cap 708. It will be understood that the vacuum chamber is also defined by the attachment of the proximal end of the probe 702 to an appropriate housing such as that shown earlier. A vacuum tight joint 710 between the cap 708 and the structure 706 can be provided by brazing or by sealing the joint with a vacuum cement or epoxy. Also shown in the Figure is the cathode needle 712 having a tip 714 from which electrons, indicated at 716, are emitted toward the anode 708.

In this embodiment of the invention shown in FIG. 7, the structure 706 may be made of insulating material with high dielectric strength, for example only and not by way of limitation, fused quartz, while the anode cap 708 may be made, for example only and not by way of limitation, of tungsten or other high nuclear number (a high Z number) metal or alloy. Alternatively, the cap 708 may comprise a low Z material having a heavy metal or high Z coating on its target surface 718, which is shown exposed to the vacuum 704 at an angle of about 45° to the path of the electron beam 716. Variation of the angle will result in changing the distribution of the x-ray beam provided by the invention as desired. That is, the angle of the target surface 718 relates to the delivery of the desired dosage of radiation since that angle defines, at least in part, the angular distribution of the x-rays emitted by the device. Thus, for different applications and different therapeutic modes of operation, the target surface 718 can lie at an angle of about 15° to about 60°.

To provide electrical contact of the cap 708 with the power supply (not shown) the outside surface 720 of the structure 706 is coated with a conductive layer 722. Layer 722 may be made as thin as desired so long as it provides the required electrical pathway. It will be understood that layer 722 will be electrically connected to the power supply at the proximal end of the probe 702 in any known, appropriate manner.

It will be understood that in the embodiment shown in the Figure, that the relative thicknesses of the structure 706 and the layer 722 have been shown wherein the structure 706 provides relatively the greatest amount of mechanical strength to the probe 702. Other relative thicknesses of the structure 706 and layer 722 may be appropriately chosen so long as the appropriate mechanical strength is provided for the particular use of such probe and so long as the relative insulative and conduction functions are also provided.

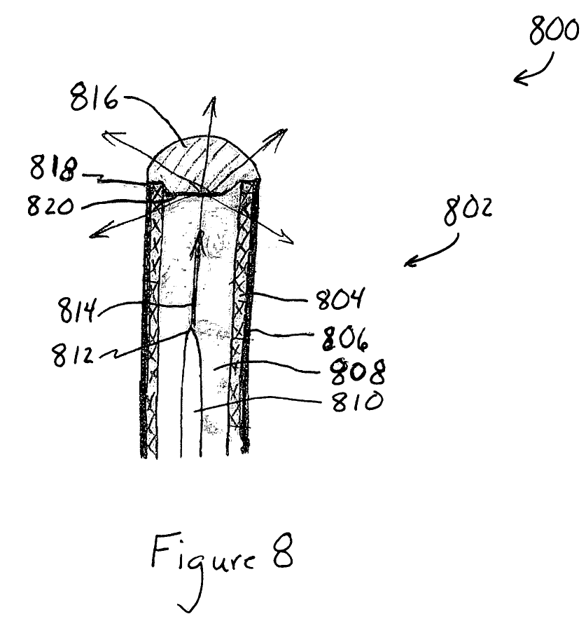

FIG. 8 illustrates another embodiment 800 of the invention that provides the emission of a generally omni-directional, close to spherical, radiation pattern. Thus, FIG. 8 shows a probe 802 comprising a conductive layer 804 and an insulative layer 806 partly defining a vacuum chamber 808. Also shown is the cathode needle 810, needle tip 812 and electron beam 814. In this embodiment an anode cap 816, attached to the probe 802 so as to provide a vacuum seal 818 as previously described, is made of low Z material for x-ray transparency, by way example only and not limitation, beryllium or pyrolytic graphite.

Pyrolytic graphite is a monocrystalline material made of carbon atoms and having a very small coefficient of thermal expansion (0.68*10−6) in planes perpendicular to the crystalline axis “c”. Thus, by providing an anode cap 816 cut from pyrolytic graphite in such a way that the crystalline axis c is oriented along the fused quartz insulating tube 806 of the probe 802, the thermal expansion of the anode cap in the radial direction makes a good match to the thermal expansion of the fused quartz (0.5*10−6) in the radial direction. In this embodiment the target surface 820 of the graphite anode cap 816 may be coated with a heavy metal, preferably tungsten, and sealingly attached to the fused quartz tube.

It is well known that electrons impinging on the target are decelerated and scattered by the atoms of the target, thereby producing x-ray radiation that propagates in all directions. Selective use of different anode materials and target thicknesses enables the production of x-ray radiation with selected directional and intensity characteristics. For example, an anode made of transparent-to-x-rays pyrolytic graphite in combination with a thin target (0.2-1.0 microns) of a heavy metal on its surface produces an omni-directional distribution of radiation. In this case the thin target stops the oncoming electrons but practically does not absorb the x-rays generated in it.

Where a directional control of the x-ray beam is desired, the target can be thickened (to more than 100 microns) to prevent propagating x-ray radiation through the target. Thus, by tailoring the geometry and thickness of the anode target and the anode materials, the effective distribution of the x-ray radiation, i.e., its preferred directional and intensity characteristics can be controlled.

Further, x-ray beam width and direction is partly dependent upon the angle of the target surface to the electron beam. A target surface, thick or thin, that is perpendicular to the electron beam will produce a generally axially symmetric x-ray radiation pattern. Angling the target surface will create an asymmetric x-ray beam in a preferred direction generally at a right angle to the electron beam, presuming the target is thick enough for preventing propagation of x-ray radiation through the target material. As the angle increases, the maximal beam width produced by the angled target will widen.

Thus, an anode capable of producing an omni-directional beam can be used, for example, in combination with a pull-back mechanism to irradiate malignant tissue or its margin after an excision of the tumor. An anode producing a beam with defined directional qualities can be used in combination with a pull-back and rotation mechanism to also irradiate malignant tissue or its margin after an excision of the tumor.

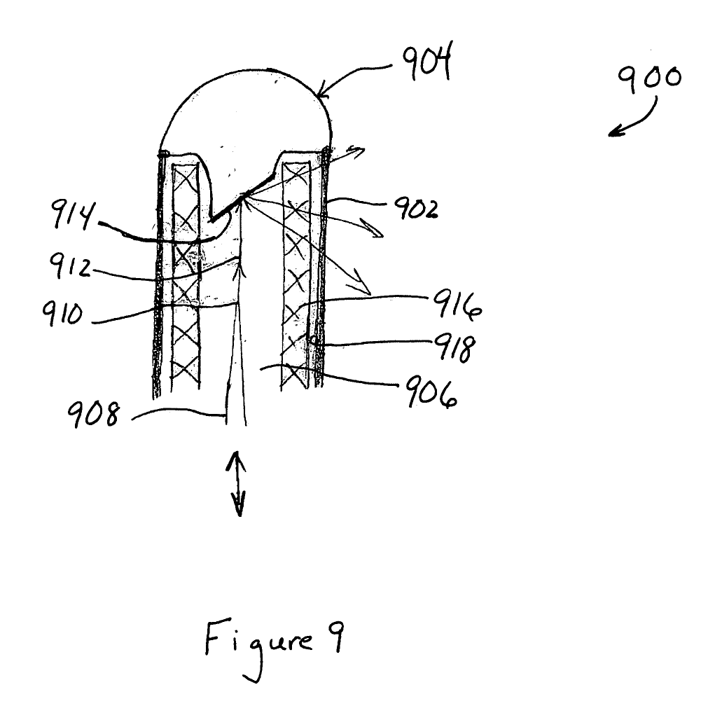

FIG. 9 illustrates another embodiment of the current invention wherein the insulative functions and conduction functions are provided by spaced apart structures. Thus, a probe 900 is shown comprising a conductive tube 902 and an anode cap 904 sealingly joined to partly define a vacuum chamber 906. A cathode needle 908 having a tip 910 emitting an electron beam 912 towards an anode target surface 914 are disposed within the chamber 906.

To insulate the cathode 908 from the tube 902 and the anode cap 904, an insulating tube, 916, preferably aligned coaxial to tube 902, is provided in the vacuum chamber 906. Thus, as shown in this embodiment, the insulating tube 916 is spaced from the cathode needle 908 as well as the inner surface 918 and the anode cap 904, and is secured in any known, appropriate manner at its proximal end. Tube 916 may be made of materials with high dielectric strength, but not necessary of high vacuum quality, for example and not by way of any limitation, boron nitride.

During use of the present invention, several Watts of electrical power will be delivered to the probe from the power supply and subsequently transferred from the cathode to the anode. The conversion of this power into x-ray radiation occurs at a low efficiency. A large portion of this electrical power is converted to heat, subjecting the probe to overheating. Thus, it is desirable to cool the probe or otherwise provide for dissipation of the generated heat to prevent overheating of the probe, and, where used for therapeutic purposes, injury to a patient.

Additionally, the conductive and insulative properties of the conductors and insulators described herein can be satisfied by any material known or hereafter developed that can perform such functions. For example, conductive polymers could be used to provide the conductive pathway provided by the conductive tube if such materials can provide the required conductance. Similarly, other insulator materials can be used in the present invention.

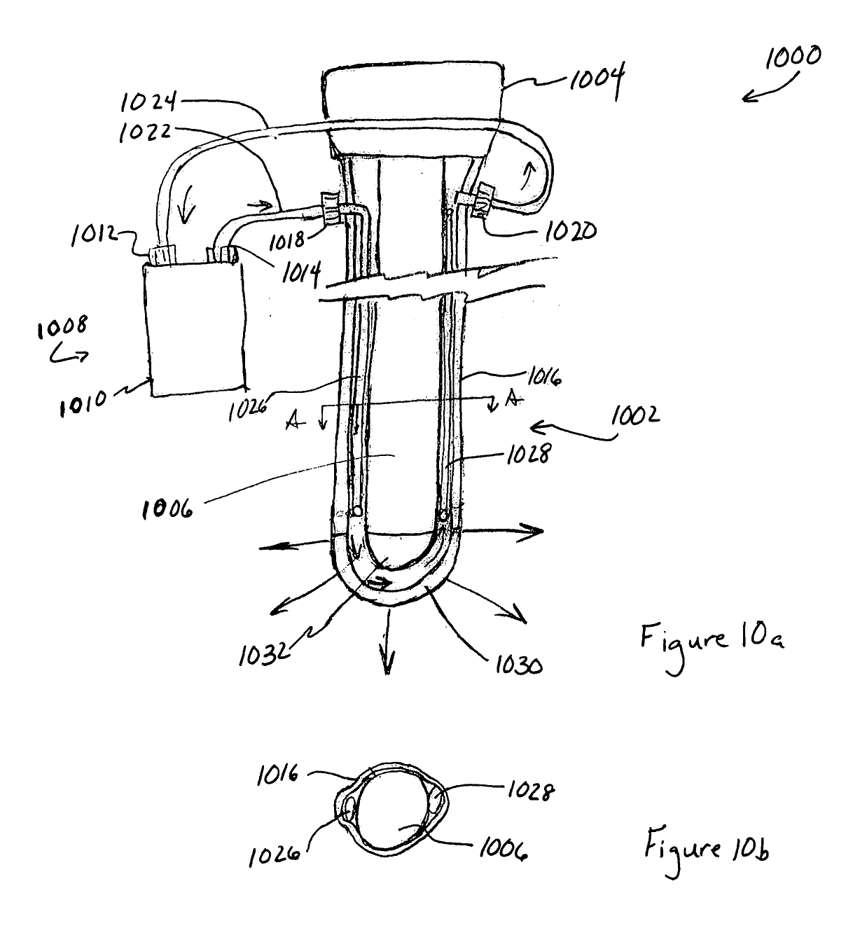

FIGS. 10a and 10b schematically shows an embodiment of the present invention 1000 that includes apparatus for dissipating the heat generated during operation thereof. X-ray apparatus 1000 includes a cooled probe 1002 and a housing 1004. The cooled probe 1002 includes a probe 1006 of the type previously described.

X-ray apparatus 1000 further comprises a cooling system 1008, including a pump 1010 having inflow and outflow ports 1012 and 1014, respectively; a cooling jacket 1016 having inflow and out jacket ports 1018 and 1020, respectively; and conduits 1022 and 1024. Conduit 1022 fluidly connects pump outflow port 1014 and jacket inflow port 1018 while conduit 1024 fluidly connects pump inflow port 1012 and jacket outflow port 1020. Cooling system 1008 may also include inflow and outflow jacket conduits 1026 and 1028, respectively, attached to the jacket inflow port 1018 and the jacket outflow port 1020. In the configuration shown in the Figure, the jacket conduits 1026 and 1028 extend from the proximal end of the probe 1006 toward the distal end thereof.

Jacket 1016 is configured to enclose cooled probe 1006. The cooling jacket 1016 may comprise a heat shrink tube that may include at its distal end 1030 a jacket end cap 1032, here configured as a dome, glued thereto so as to encompass the anode 1034 of the cooled probe 1002. Jacket end cap 1032 should be made of a material transparent to x-rays.

In operation, a coolant fluid is pumped from the pump 1010 from a reservoir (not shown) through the conduit 1022 into the jacket inflow conduit 1026. The coolant fluid flows around the probe 1006 generally and particularly around the outer surface of the anode 1032 in a good thermal contact with it to thereby remove the heat deposited during operation of the invention. After thermal interaction with the anode the coolant fluid proceeds into the jacket outflow conduit 1028 and back to the pump via the conduit 1024.

If desired, the jacket outflow conduit 1028 may be omitted from the system 1008. Without it, the coolant fluid flows from the vicinity of the anode 1032 through the annular space around the probe to the jacket outflow port and then back to the pump 1010 as previously describe.

Any fluid, gas or liquid, capable of providing the necessary thermal transference from the anode 1032 may be used. If a liquid coolant is used, the volume of the liquid coolant required for efficient cooling of the anode falls into a range of 10-100 cc/min. The temperature of the incoming coolant may lay in a range from a room temperature 20° C. (preferably) up to a body temperature 37° C.

Referring particularly to FIG. 10b, a cross-section of FIG. 10a taken along cutting plane A-A is shown. For ease of showing the cooling system portion of the present invention, the probe 1006 has not been shown. It will be observed that the jacket 1016 is configured to closely conform to the shape of the probe 1006, with the required space needed for the conduits 1026 and 1028 being made along opposite sides thereof. This configuration reduces the overall cross-sectional area of the device, facilitating its use.

The x-ray device of the present invention can also be used for radiation treatments of inoperable tumors, for example, certain tumors in the brain. In this case, when only a narrow channel through the tumor can be made (in part for taking a biopsy test), the best mode of treatment can be provided by a version of the current invention in which a one-side radial radiation beam is utilized in combination with a pull-back and rotation of the probe around its axis. Guided by a 3-D image of the irradiation target zone, this mode of the treatment is capable of delivering a predetermined radiation dose to a treatment zone of practically any shape.

It is widely accepted among radiation oncologists that when a surgical removal of a tumor is possible, the best mode of radiation treatment is an intra-operative one with irradiation of the margin tissue around the excised tumor performed shortly and preferably immediately after the surgical removal of the tumor. In current treatment therapies of soft tissue tumors, like those found in the breast or brain, an inflatable balloon is placed into the cavity created by the removal of the tumor and is inflated. The balloon is configured and structured to stretch the cavity to a known shape, preferably spherical, elliptical or cylindrical. The target zone for radiation treatment is thus established as the marginal tissue around the balloon to a depth in the tissue of about 1 cm. The advantage of this approach is that the target zone for irradiation becomes a simple and symmetrical one that easily can be covered with the existing patterns of ionizing radiation produced by a radiation source based on radioactive materials or x-ray emitters.

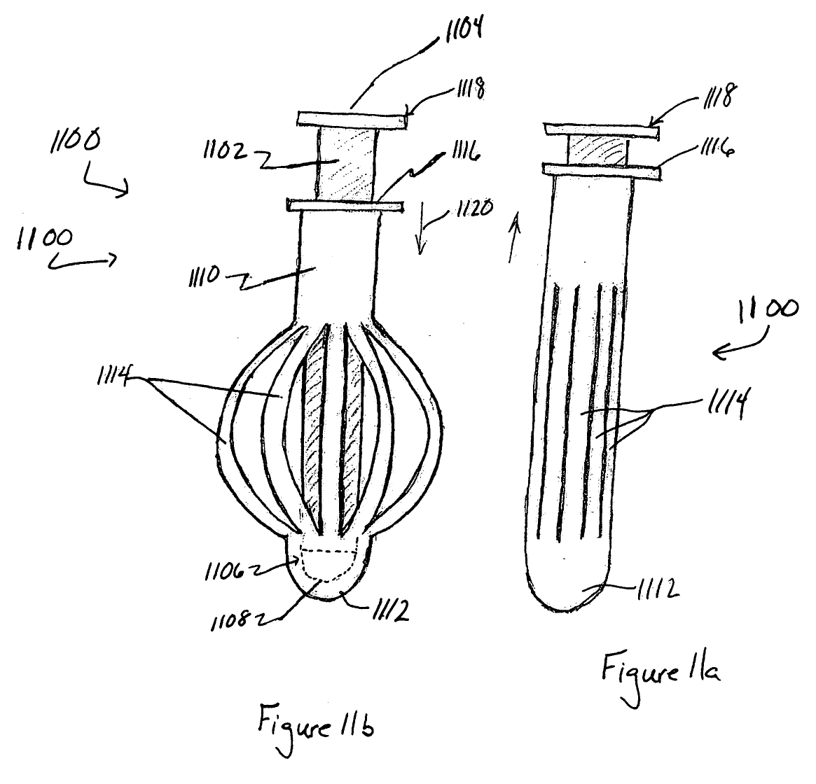

FIGS. 11a and 11b illustrates a novel appliance 1100 for stretching the cavity created by surgical excision of a tumor. FIG. 11a depicts the appliance in its relaxed or compressed position for insertion into the cavity remaining after the tumor excision while FIG. 11b shows the appliance in its expanded position for stretching the tissue surrounding an excised tumor. Appliance 1100 comprises a first or inner hollow shaft 1102 configured to receive an x-ray apparatus as described herein from its open proximal end 1104. The distal end 1106 of the hollow shaft 1102 is hermetically sealed with a dome 1108, which may be integral with the shaft 1102. A second or outer hollow shaft 1110 having a distal end 1112 is configured to coaxially receive the first shaft 1102. The first and second shafts 1102 and 1110 are secured to each other at their respective distal ends 1106 and 1112 by any known manner, such as but not limited to gluing.

Second shaft 1110 includes a plurality of flexible ribs 1114. Because of the affixation of the distal ends 1106 and 1112, the proximal end 1116 of the second shaft 1110 can moved back and forth relative to the proximal end 1118 of the first shaft 1102. In the position shown in the FIG. 1 a the ribs 1114 have significantly lower profile. Movement of the second shaft proximal end 1116 away from the first shaft proximal end 1118 as indicated by arrow 1120 in FIG. 11b forces the ribs 1114 to flex outwardly away from the shafts to adopt a high profile position.

As shown in FIG. 11a, appliance 1100 is adapted for insertion into the cavity left after surgical excision of a tumor. After insertion of the appliance into tissue the cavity the appliance is secured in the high profile state by movement of the second shaft proximal end 1116 as described, in which the ribs 1114 stretch the tissue around the incision to a predetermined symmetric shape adapted for irradiation. With this appliance, a rotation of the x-ray emitting probe is not required; a pull back mode of an x-ray emitting probe with an axially symmetric radiation pattern is adequate.

Appliance 1100 may be made of any material that provides the functions recited herein, such as plastic or metal.

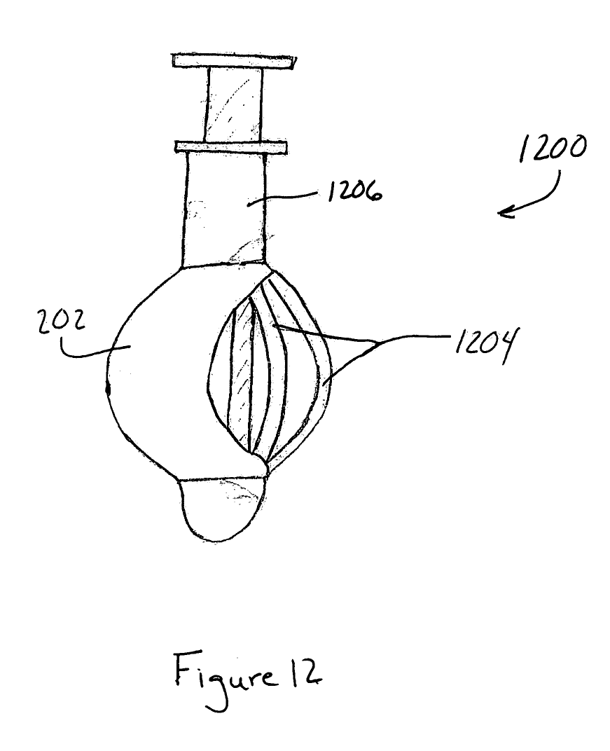

Another embodiment of an appliance 1200 is shown in FIG. 12. Appliance 1200 includes a compliant or flexible skin 1202 secured over the ribs 1204 of the appliance 1200. The skin 1202 is hermetically sealed to the outer hollow shaft 1206 of the appliance.

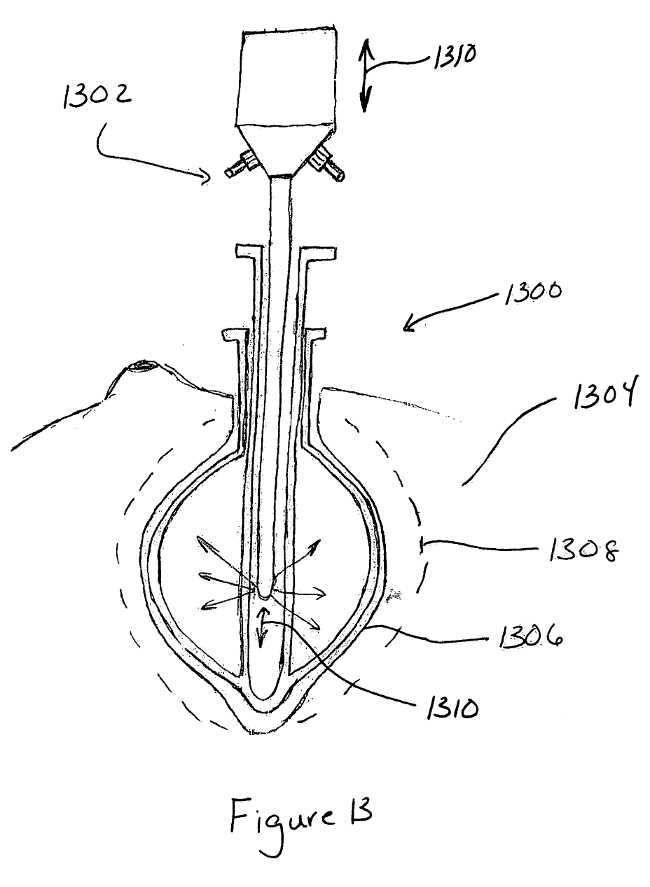

FIG. 13 illustrates an example of the use of an appliance 1300 of the type shown in FIGS. 11-12 in combination with an x-ray apparatus 1302 as described herein for an irradiation treatment following surgical excision of a breast tumor. Thus the Figure shows a breast 1304 having a cavity 1306 created by the excision of a tumor. The cavity 1306 has been expanded by the use of the appliance 1300 as previously described. A target zone for depth of irradiation of the margin tissue surrounding the tumor is indicated by the dotted line 1308. As indicated by double headed arrow 1310, the apparatus 1302 is movable back and forth relative to the cavity 1306, facilitating irradiation of the target zone. As noted earlier, the appliance 1300 would be inserted into the cavity 1306 with the appliance in the position shown in FIG. 1 la and would then be placed into the expanded or tissue stretching configuration shown in FIG. 13. If desired, the appliance can be inserted alone and then the apparatus 1302 may be inserted therein, or the apparatus 1302 may first be placed with the appliance and the appliance then inserted. The use in the Figure contemplates the use of either form of the appliance shown in FIGS. 11 or 12.

The present invention has been described relative to several specific and various embodiments and procedures for use. Those skilled in the art will recognize that certain features described herein can be interchanged with other known devices. For example, but not limited thereto, adjustment of the vacuum gap has been accomplished by translational movement of the needle cathode (FIG. 1, for example) or the needle anode (FIG. 3, for example) by use of a step motor or inchworm linear motor. Any device, however, that can provide the desired translational movement with the requisite precision and size may be used with equal facility.

As a further example, where layered structures are described, such as the tube 706 and coating layer 722 of FIG. 7, it should be understood that any combination of suitable insulator/conductor materials may be used. Additionally, such layered structures may take on varying dimensions where only one or both provides the required mechanical strength for operation of the present invention. Thus, by way of example only, the tube 706 of FIG. 7 may provide the required mechanical strength while the layer 722 serves only to provide a conductive pathway. Alternatively, both the tube 706 and the layer 722 may be configured to provide mechanical strength or the layer 722 may be configured to provide required mechanical strength alone while the tube 706 serves primarily only an insulative function. Those skilled in the art will recognize other such variations relative to other embodiments described herein.

The present invention has been described in language more or less specific as to the apparatus and method features illustrated in the Figures. It is to be understood, however, that the present invention is not limited to the specific features described, since the apparatus and method herein disclosed comprise exemplary forms of putting the present invention into effect. For example, while the invention has been described relative to uses in the medical therapy field, it could find advantageous use whenever a field emission x-ray apparatus is used for any other purpose. The invention is, therefore, claimed in any of its forms or modifications within the proper scope of the appended claims appropriately interpreted in accordance with the doctrine of equivalency and other applicable judicial doctrines.

1. An apparatus for producing x-rays with operating current control comprising:
a housing;
a probe, said probe including:
distal and proximal ends, with said proximal end being attached to said housing; and
an outer wall structure defining a hollow interior;
an anode attached to said probe at said probe distal end;
a cathode having proximal and distal ends, wherein:
said distal cathode end is disposed within said distal probe end and spaced from said anode to create a gap therebetween; and
said proximal cathode end is disposed within said housing; and
a linear translator attached to said proximal cathode end and being provided for moving said cathode toward and away from said anode to adjust said gap and change the operating current;
wherein said probe comprises a conductor and said cathode is insulated from said conductor by an insulator therebetween.

2. The apparatus of claim 1 wherein said insulator engages said conductor.

3. The apparatus of claim 1 wherein said insulator is spaced from said conductor and said cathode.

4. The apparatus of claim 1 wherein said probe defines an axis and said anode includes a target surface disposed at an angle of about 15° to about 60° relative to said probe axis.

5. The apparatus of claim 1 wherein said anode is made of pyrolytic graphite and said anode includes a target surface formed from a high nuclear number material.

6. The apparatus of claim 5 wherein said insulator is a high strength dielectric.

7. The apparatus of claim 5 wherein said insulator is fused quartz.

8. The apparatus of claim 1 wherein said insulator is a high strength dielectric.

9. The apparatus of claim 1 and further including a cooling system, said cooling system comprising:
a pump for pumping a coolant fluid;
a jacket having a hollow interior configured to receive said probe; and
conduits connecting said pump to said jacket;
wherein said pump circulates a coolant fluid through said jacket to cool said probe during operation thereof.

10. The apparatus of claim 9 and further including an appliance for stretching tissue, said appliance comprising:
a first hollow shaft having first shaft proximal and distal ends, said first hollow shaft configured to receive said probe;
a second hollow shaft having second shaft proximal and distal ends, said second hollow shaft configured to receive said first shaft, said second hollow shaft distal end being secured to said first hollow shaft distal end, said second hollow shaft including a plurality of flexible ribs configured to flex outwardly when said second hollow shaft proximal end is moved toward said second hollow shaft distal end.

11. An apparatus for providing producing x-rays with operating current control comprising:
a housing;
a probe, said probe including:
distal and proximal ends, with said proximal end being attached to said housing; and
an outer wall structure defining a hollow interior, said outer wall structure including an outer conductive layer and an inner insulative layer;
an anode attached to said probe at said probe distal end;
a cathode having proximal and distal ends, wherein:
said distal cathode end is disposed within said distal probe end and spaced from said anode to create a gap therebetween; and
said proximal cathode end is disposed within said housing; and
a linear translator attached to said proximal cathode end and being provided for moving said cathode toward and away from said anode to adjust said gap and change the operating current.

12. The apparatus of claim 11 wherein said probe defines an axis and said anode includes a target surface disposed at an angle of about 15° to about 60° relative to said probe axis.

13. The apparatus of claim 11 wherein said anode is made of pyrolytic graphite and said anode includes a target surface formed from a high nuclear number material.

14. The apparatus of claim 13 wherein said insulator is a high strength dielectric.

15. The apparatus of claim 11 wherein said insulator is a high strength dielectric.

16. The apparatus of claim 11 and further including a cooling system, said cooling system comprising:
a pump for pumping a coolant fluid;
a jacket having a hollow interior configured to receive said probe; and
conduits connecting said pump to said jacket;
wherein said pump circulates a coolant fluid through said jacket to cool said probe during operation thereof.

17. The apparatus of claim 16 and further including an appliance for stretching tissue, said appliance comprising:
a first hollow shaft having first shaft proximal and distal ends, said first hollow shaft configured to receive said probe;
a second hollow shaft having second shaft proximal and distal ends, said second hollow shaft configured to receive said first shaft, said second hollow shaft distal end being secured to said first hollow shaft distal end, said second hollow shaft including a plurality of flexible ribs configured to flex outwardly when said second hollow shaft proximal end is moved toward said second hollow shaft distal end.

18. A method for providing x-ray radiation therapy to a patient having a tumor, said method comprising:
excising the tumor to create a body cavity;
disposing within the body cavity an appliance for stretching the body cavity to a desired configuration;
stretching the body cavity to the desired configuration using the appliance to create a stretched body cavity;
providing an x-ray apparatus for emitting x-rays for x-ray therapy, the apparatus including an elongate probe with a distal end including an anode and a cathode separated by gap;
disposing the apparatus within the stretched body cavity;
irradiating the target with x-rays produced by the apparatus;
monitoring the operating current of the x-ray apparatus; and adjusting the gap between the anode and the cathode at least once to stabilize the x-ray emission to provide the desired irradiation dose rate for the therapy.

19. The method of claim 18 wherein the appliance comprises:
a first hollow shaft having first shaft proximal and distal ends, said first hollow shaft configured to receive said probe;
a second hollow shaft having second shaft proximal and distal ends, said second hollow shaft configured to receive said first shaft, said second hollow shaft distal end being secured to said first hollow shaft distal end, said second hollow shaft including a plurality of flexible ribs configured to flex outwardly when said second hollow shaft proximal end is moved toward said second hollow shaft distal end.
